(19) United States
(12) Patent Application Publication
(10) Pub. No.: US 2017/0135947 A1
(43) Pub. Date: May 18, 2017

(54) Hair-Applicable Composition to Visually Identify a Single Child amongst Other Children in a Public Setting

(71) Applicant: Regina Picciano, Mahopac, NY (US)

(72) Inventor: Regina Picciano, Mahopac, NY (US)

(21) Appl. No.: 14/942,321

(22) Filed: Nov. 16, 2015

(51) Int. Cl. A61K 8/96; A61Q 17/04; A61K 8/04; A61Q 5/06; A61K 8/02

(52) U.S. Cl. CPC A61K 8/96; A61Q 5/065; A61K 8/0216; A61K 8/042; A61K 8/046; A61Q 17/04; A61K 2800/43; A61K 2800/591

(57) ABSTRACT

A hair-applicable composition and method for visually identifying a single child amongst other children engaging in water-related activities or in another public setting. The composition includes a hair-application base and a waterproof hair dye. The hair-application base is the medium by which the waterproof hair dye is applied to the hair of a child. The waterproof hair dye includes a visually-vibrant pigmenting agent and a hydrophobic enabling agent. The visually-vibrant pigmenting agent alters the hair color of the child in order to allow a supervisor to easily identify him or her. The hydrophobic enabling agent ensures the color does not wash out with water when the child is engaging in water-related activities. The waterproof hair dye is homogeneously mixed into the topical base to create a latherable homogeneous composition.

### FIELD OF THE INVENTION

The present invention relates generally to a composition for visually identifying children in water-related activities. More specifically, the present invention is waterproof hair dye which improves the supervision of children in water-related activities. The present invention is temporary, easy to wash off, non-toxic, and environmentally friendly.

### BACKGROUND OF THE INVENTION

Pools, lakes, ponds, and beaches mean summer fun and cool relief from hot weather. But water also can be dangerous for kids if the proper precautions are not taken. Drowning is a major concern for any individuals participating in water-related activities. This concern is mostly related towards kids as they most often times do not know how to swim, and they can drown in less than two feet water because of their size. Nearly 1,000 kids die each year by drowning, with most of these deaths occurring in home swimming pools. It is the second leading cause of accidental death for people between the ages of five and 24. To reduce the chances for drowning, a few safety precautions should be taken. These include swimming lessons, floatation devices, and swimming near lifeguards. The most important and most effective precaution that any parent could implement is constant supervision. All kids need supervision in water-related activities, regardless of swimming skills This can be hard in public areas where the presence of people makes it hard to spot your own child. Some possible solutions include colored swim caps, specific hair styles, and color matching bathing suits. Unfortunately, each of these solutions is limited in their approach; no kid ever wants to wear a swim cap, not all kids have the same length hair, and parents cannot see bathing suits under water.

The present invention is a method and composition for improving the visibility and identification of kids in public, in particular in and around water. The present invention is a waterproof hair dye which allows parents or groups to color code themselves and their kids in order to easily keep track of them while engaging in water-related activities. The present invention comes in a variety of colors for both boys and girls. Additionally, the present invention is easily removable with shampoo, does not damage the hair, is environmentally friendly, and does not stain clothes.

### BRIEF DESCRIPTION OF THE DRAWINGS

FIG. 1 is a block diagram depicting the ingredients of the present invention.

FIG. 2 is a flowchart depicting the overall process of the present invention.

FIG. 3 is a flowchart depicting the sub-steps of applying the present invention as cream or gel onto the child's head.

FIG. 4 is a flowchart depicting the sub-steps of applying the present invention as an aerosol spray onto the child's head.

FIG. 5 is a flowchart depicting how the present invention visually marks the child.

FIG. 6 is a flowchart depicting how to wash out the present invention from the child's head.

FIG. 7 is a flowchart depicting how to protect the child's head from ultraviolet rays with the present invention.

### DETAIL DESCRIPTIONS OF THE INVENTION

All illustrations of the drawings are for the purpose of describing selected versions of the present invention and are not intended to limit the scope of the present invention.

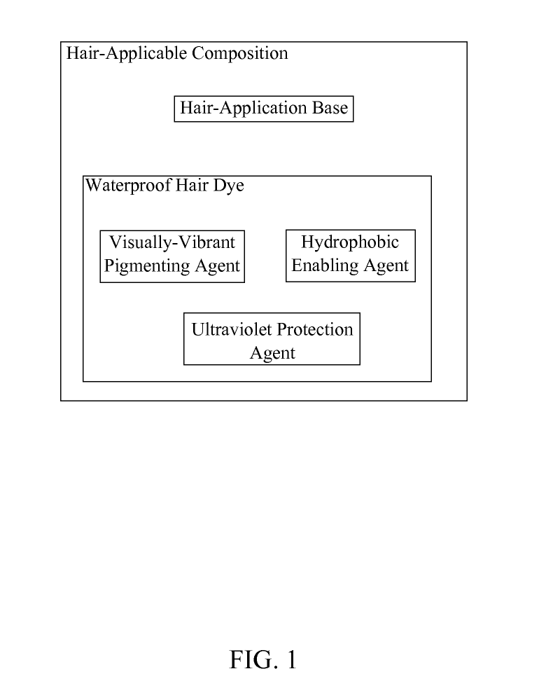

The present invention is a hair-applicable composition to visually distinguish a single child playing amongst other children in some kind of public setting. The hair-applicable composition is specifically configured to not wash out of a child's hair while the child plays in water or engages in other water-related activities. However, the present invention is also useful in other kinds of public settings such as a sporting event because the present invention does not wash out from the child's sweat. Consequently, the present invention comprises a hair-application base and a waterproof hair dye, which is shown in FIG. 1. The hair-application base allows the user to uniformly apply the waterproof hair dye throughout the head hair of a child as a way to visually mark the top of the child's head. The user can be anyone from a parent or guardian of the child or a caretaker. Thus, the waterproof hair dye is homogeneously mixed into the hair-application base. Once the present invention is applied onto the head hair, the hair-application base is either removed from the head hair, dried out of the head hair, or dissipated out of the head hair, which leaves only the waterproof hair dye on the head hair. The waterproof hair dye alters the visual appearance of the child so that the user is able to easily identify the child amongst other children. The waterproof hair dye does not wash away in water, which allows the child to play in water and allows the user to visually distinguish and keep track of the child.

The hair-application base is used to apply and to distribute the waterproof hair dye throughout the head hair of the child. The hair-application base can be, but is not limited to, a cream-type substance, a gel-type substance, or an aerosol substance. If the hair-application base is a cream-type substance, then the present invention could also act as an emollient to moisturize the child's scalp. If the hair-application base is a gel-type substance, then the present invention could also have a soothing effect on the child's scalp. If the hair-application base is an aerosol substance, then the present invention is able to rapidly separate the hair-application base from the waterproof hair dye so that the present invention can be faster applied to the head hair of the child.

The waterproof hair dye allows the user to visually mark and easily identify the child while the child plays in the water or is involved in other kinds of water-related activities. The waterproof hair dye includes, is but not limited to, the following composition components: a visually-vibrant pigmenting agent, a hydrophobic enabling agent, and an ultraviolet protection agent. The visually-vibrant pigmenting agent is used to change to the color of the head hair to a recognizable, bright color such as yellow, pink, orange, or red. The hydrophobic enabling agent is used to prevent the visually-vibrant pigmenting agent from washing out of the head hair when the waterproof hair dye comes into contact with water. The ultraviolet protection agent is used to prevent sunburn on the child's scalp, which is a situation that commonly arises while a child plays near a body of water on a sunny day.

The waterproof hair dye also needs to be a temporary hair dye such that the user or the child may wash the waterproof hair dye out of the head hair after the child is done playing in the water. Consequently, the waterproof hair dye needs to be molecularly configured to selectively bond to shampoo compounds over water molecules. This molecular configuration allows the waterproof hair dye to be washed out with a combination of water and shampoo but not washed out with water by itself.

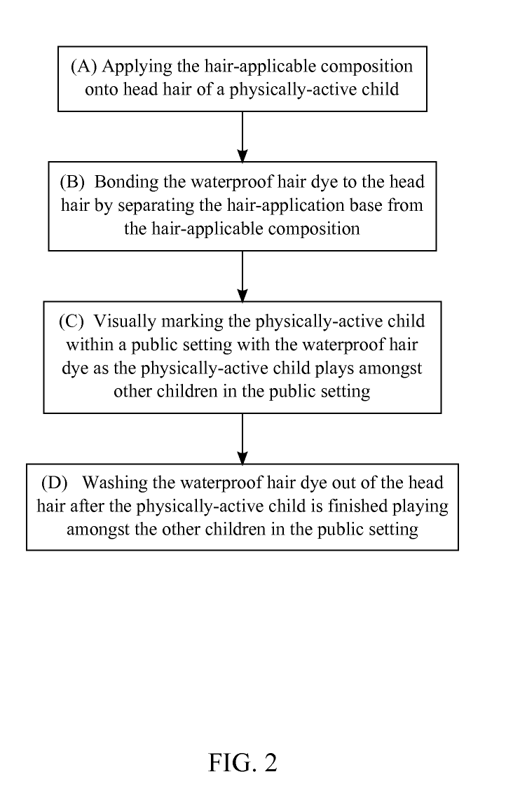

As can be seen in FIG. 2, the method of using the present invention includes the following steps. The method begins by providing the present invention as a hair-applicable composition and by applying the hair-applicable composition onto the head hair of a physically-active child. The physically-active child is a child that is more likely to swim and/or to run-around a public setting and, consequently, is more likely to get lost amongst other children. In addition, the waterproof hair dye is applied to the head hair because the top of the child's head is the most easily distinguishable part of the child. More specifically, the waterproof hair dye is distributed throughout the head hair by pushing the hair-application base through the head hair.

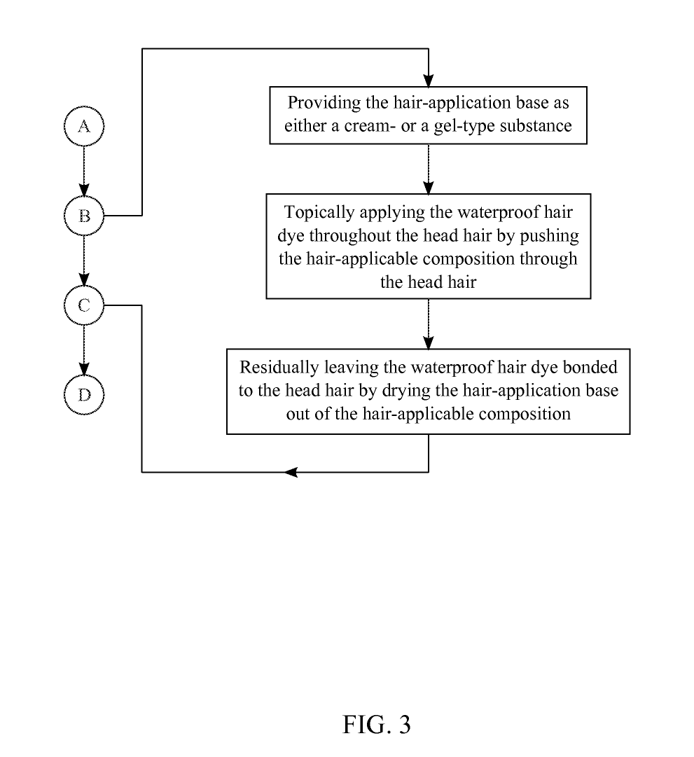

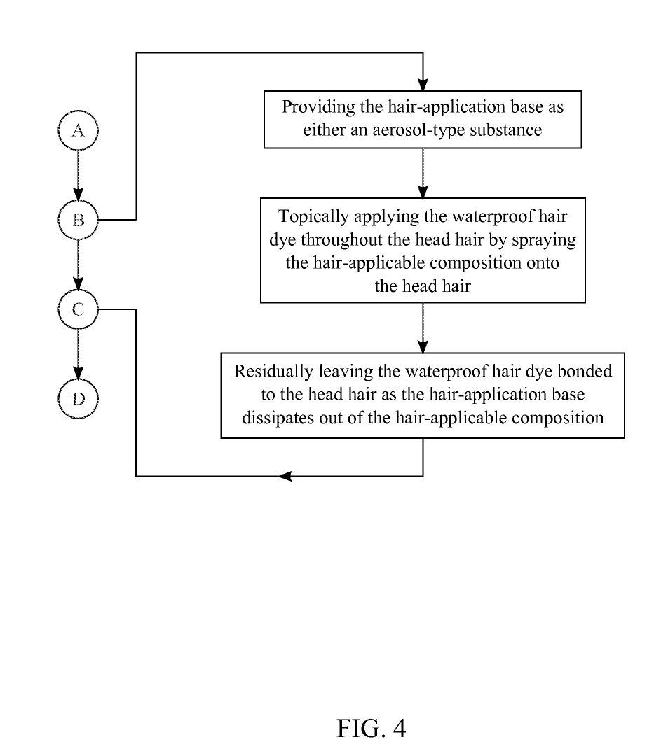

The method proceeds by bonding the waterproof hair dye to the head hair by separating the hair-application base from the hair-applicable composition. This allows the waterproof hair dye to be temporarily affixed to the head hair. In one scenario shown in FIG. 3, the hair-application base is either a cream-type substance or a gel-type substance, and the waterproof hair dye is topically applied throughout the head hair by physically pushing the hair-application composition through the head hair. In order to separate the hair-application base from the topical hair-applicable composition, the waterproof hair dye is residually left bonded to the head hair as the hair-application base is dried out of the hair-applicable composition. In another scenario shown in FIG. 4, the hair-application base is an aerosol substance, and the waterproof hair dye is topically applied throughout the head hair by spraying the hair-application composition onto the head hair. In order to separate the hair-application base from the topical hair-applicable composition, the waterproof hair dye is residually left bonded to the head hair as the hair-application base dissipates out of the hair-applicable composition.

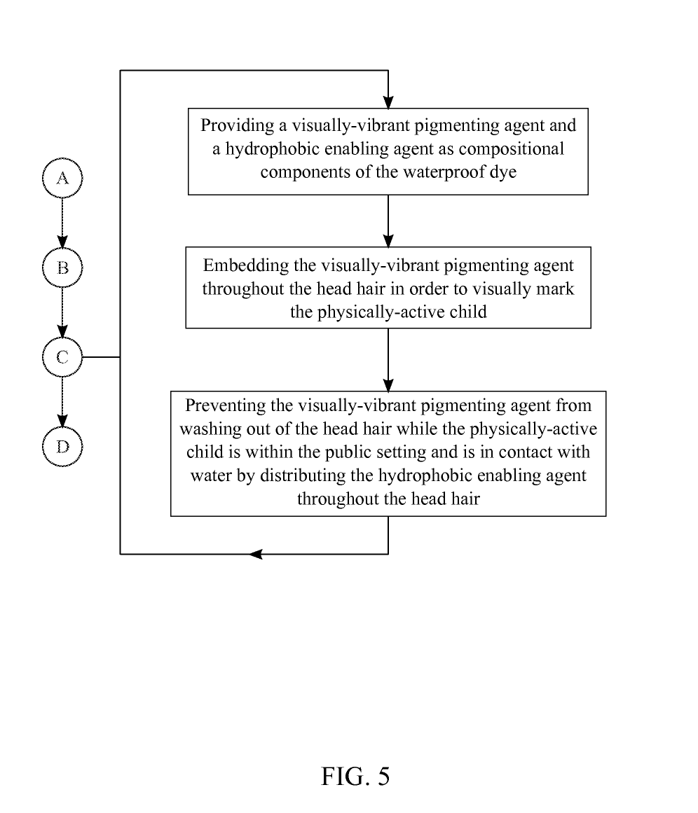

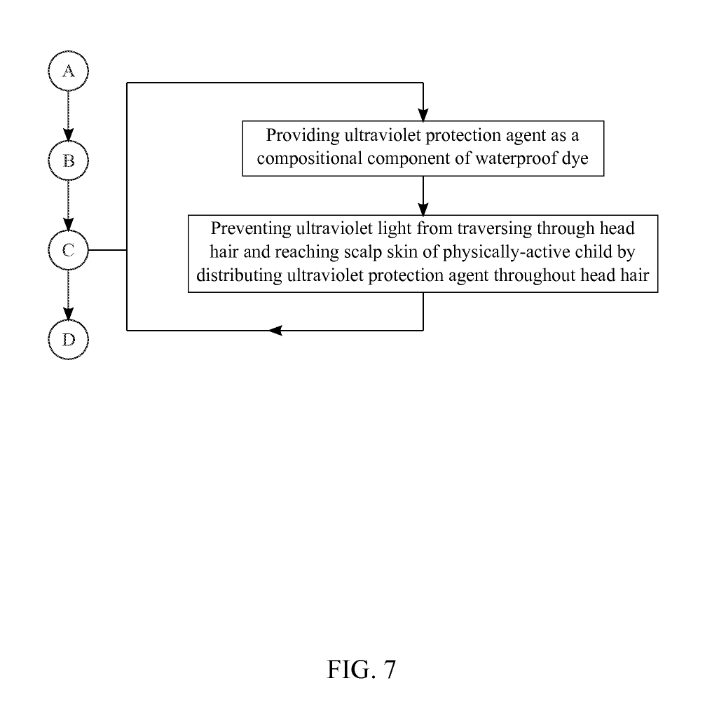

When the physically-active child enters a public setting, the method of using the present invention continues by visually marking the physically-active child within the public setting with the waterproof hair dye as the physically-active child plays amongst the other children in the public setting. The public setting can be any place with a lot of children such as near a beach, a pool, a playground, or a sports field. This allows the user to distinguish and watch over the physically-active child, even when the physically-active child is playing in a body of water or profusely sweating to where a normal hair dye would run off the head hair of the child. More specifically, the visually-vibrant pigmenting agent is embedded throughout the head hair in order to visually mark the physically-active child, and the visually-vibrant pigmenting agent is prevented from being washed out of the head hair while the physically-active child is within a body of water or is profusely sweating because the hydrophobic enabling agent is distributed throughout the head hair. These steps are shown in FIG. 5. The method of using the present invention also prevents ultraviolet light from traversing through the head hair and the reaching the scalp skin of the physically-active child. As can be seen from the steps in FIG. 7, this occurs because the ultraviolet protection agent is also distributed throughout the head hair.

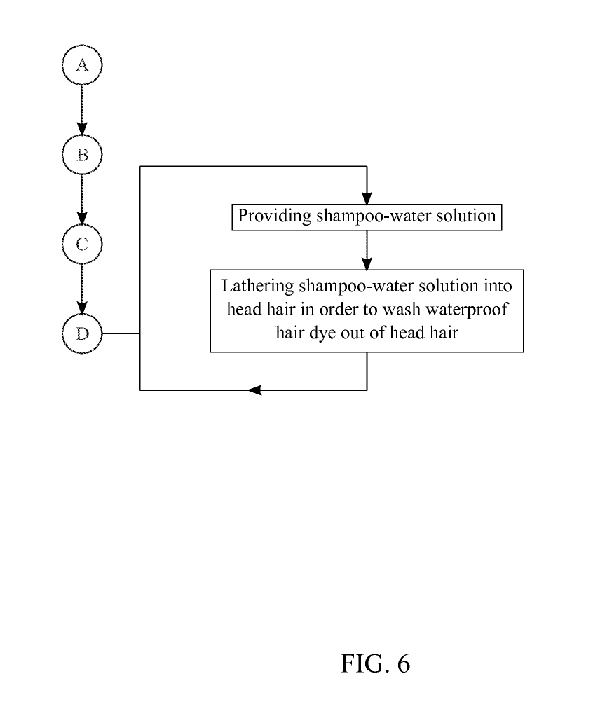

Finally, the method of using the present invention is completed by washing the waterproof hair dye out of the head hair after the physically-active child is finished playing amongst the other children in the public setting, which is shown in FIG. 6. More specifically, a shampoo-water solution is provided to the user or the child, and the shampoo-water solution is lathered into the head hair in order to watch the hair dye out of the head hair.

Although the invention has been explained in relation to its preferred embodiment, it is to be understood that many other possible modifications and variations can be made without departing from the spirit and scope of the invention as hereinafter claimed.

What is claimed is:

1. A hair-applicable composition to visually distinguish a single child playing amongst other children comprises:
a hair-application base;
a waterproof hair dye;
the waterproof hair dye comprises a visually-vibrant pigmenting agent and a hydrophobic enabling agent; and
the waterproof hair dye being homogeneously mixed into the hair-application base.

2. The hair-applicable composition to visually distinguish a single child playing amongst other children as claimed in claim 1, wherein the hair-application base is a cream-type substance.

3. The hair-applicable composition to visually distinguish a single child playing amongst other children as claimed in claim 1, wherein the hair-application base is a gel-type substance.

4. The hair-applicable composition to visually distinguish a single child playing amongst other children as claimed in claim 1, wherein the hair-application base is an aerosol substance.

5. The hair-applicable composition to visually distinguish a single child playing amongst other children as claimed in claim 1 comprises:
the waterproof hair dye further comprises an ultraviolet protection agent.

6. The hair-applicable composition to visually distinguish a single child playing amongst other children as claimed in claim 1, wherein the waterproof hair dye is molecularly configured to selectively bond to shampoo compounds over water molecules.

7. A method of using the hair-applicable composition to visually distinguish a single child playing amongst other children as claimed in claim 1, the method comprises the steps of:
applying the hair-applicable composition onto head hair of a physically-active child;
bonding the waterproof hair dye to the head hair by separating the hair-application base from the hair-applicable composition;
visually marking the physically-active child within a public setting with the waterproof hair dye as the physically-active child plays amongst other children in the public setting; and
washing the waterproof hair dye out of the head hair after the physically-active child is finished playing amongst the other children in the public setting.

8. The method as claimed in claim 7 comprises the steps of:
providing the hair-application base as either a cream-type substance or a gel-type substance; and
topically applying the waterproof hair dye throughout the head hair by pushing the hair-applicable composition through the head hair.

9. The method as claimed in claim 7 comprises the step of:
residually leaving the waterproof hair dye bonded to the head hair by drying the hair-application base out of the hair-applicable composition.

10. The method as claimed in claim 7 comprises the steps of:
providing the hair-application base as an aerosol substance; and
topically applying the waterproof hair dye throughout the head hair by spraying the hair-applicable composition onto to the head hair.

11. The method as claimed in claim 7 comprises the step of:
residually leaving the waterproof hair dye bonded to the head hair as the hair-application base dissipates out of the hair-applicable composition.

12. The method as claimed in claim 7 comprises the steps of:
providing a visually-vibrant pigmenting agent and a hydrophobic enabling agent as compositional components of the waterproof dye;
embedding the visually-vibrant pigmenting agent throughout the head hair in order to visually mark the physically-active child; and
preventing the visually-vibrant pigmenting agent from washing out of the head hair while the physically-active child is within the public setting and is in contact with water by distributing the hydrophobic enabling agent throughout the head hair.

13. The method as claimed in claim 7 comprises the steps of:
providing a shampoo-water solution; and
lathering the shampoo-water solution into the head hair in order to wash the waterproof hair dye out of the head hair.

14. The method as claimed in claim 7 comprises the step of:
providing an ultraviolet protection agent as a compositional component of the waterproof dye; and
preventing ultraviolet light from traversing through the head hair and reaching scalp skin of the physically-active child by distributing the ultraviolet protection agent throughout the head hair.
